(19) United States
(12) Patent Application Publication
(10) Pub. No.: US 2002/0183549 A1
(43) Pub. Date: Dec. 5, 2002

(54) Method and apparatus of methyl acetate hydrolysis

(76) Inventor: Myron M. Lee, Seongnam-City (KR)

Correspondence Address: Eugene Oak, 610 S. Van Ness Ave., Los Angeles, CA, 90005, US

(21) Appl. No.: 09/878,153

(22) Filed: Jun. 11, 2001

(30) Foreign Application Priority Data
May 12, 2000 (KR) 10-2000-0039788

(51) Int. Cl. C07C 53/08

(52) U.S. Cl. 562/607

(57) ABSTRACT

A novel process of methyl acetate hydrolysis equipped with a fixed catalyst bed is invented. Methyl acetate catalytically reacts with water in a liquid phase to produce acetic acid and methanol. The reactor effluent is transferred to a distillation column, which separates unreacted methyl acetate from the reaction products. The vaporized methyl acetate is condensed and recycled to the reactor. The reflux drum of the distillation column is used as the fixed bed reactor, which is packed with the solid acid catalysts. The reaction products, acetic acid and methanol, are transferred to another distillation column for a separation of methanol. The process of this invention enables reduction of investment and operation costs compared to the conventional hydrolysis and reactive distillation processes.

The present invention relates to a novel process of hydrolyzing methyl acetate to produce acetic acid and methanol.

### BACKGROUND OF THE INVENTION

1. Field of Invention

This invention relates to a novel process in that methyl acetate is reacted with water to produce acetic acid and methanol in a liquid-phase reactor equipped with a fixed bed of solid acid catalysts.

2. Description the Prior Art

Methyl acetate is produced in large quantities as a by-product from many chemical processes of, including but not limited to, terephthalic acid, isophthalic acid, polyvinyl alcohol, and trimellitic anhydride production. Due to the high volatility and low economic value of impure methyl acetate, it is usually discharged into the atmosphere after scrubbing and a wastewater treatment system or burned in an incinerator.

A conventional process for the methyl acetate hydrolysis requires a large excess of water in order to increase the conversion efficiency of methyl acetate. Due to a reversible reaction and a low equilibrium constant, the reaction products contain all four components and a complicated separation scheme with three or four distillation columns are required. As a result, the process is not economical and the unfavorable process economics have prevented its wide applications in the commercial plants.

Recently, a reactive distillation process in that both reaction and separation are carried out in the same column has been developed to improve the process economics. Since both the reaction and the separation occur in the liquid and the gaseous phase, however, the new process has some drawbacks such as lower mass transfer efficiencies, relatively large volume of the reaction zone and difficulties of packaging catalysts. Hence, the investment cost is relatively high and expensive catalysts are susceptible to erosion and abrasion.

### SUMMARY OF THE INVENTION

The purpose of this invention is to provide a novel and more economical process by eliminating and minimizing the disadvantages of both the conventional and the reactive distillation process of methyl acetate hydrolysis. Methyl acetate is catalytically reacted with water in a liquid phase to produce acetic acid and methanol. The reactor effluent is transferred to a distillation column, which separates unreacted methyl acetate from the reaction products. The vaporized methyl acetate is condensed and recycled to the reactor. The reflux drum of the distillation column is used as the fixed bed reactor, which is packed with solid acid catalysts. The reaction products, acetic acid and methanol, are transferred to another distillation column for separation of methanol. The process of this invention enables reduction of investment and operation costs compared with the conventional hydrolysis and reactive distillation processes.

### BRIEF DESCRIPTION OF FIGURES

FIG. 1 is a schematic flow diagram of the process of this invention.

### DETAILED DESCRIPTION OF THE PREFERRED EMBODIMENTS

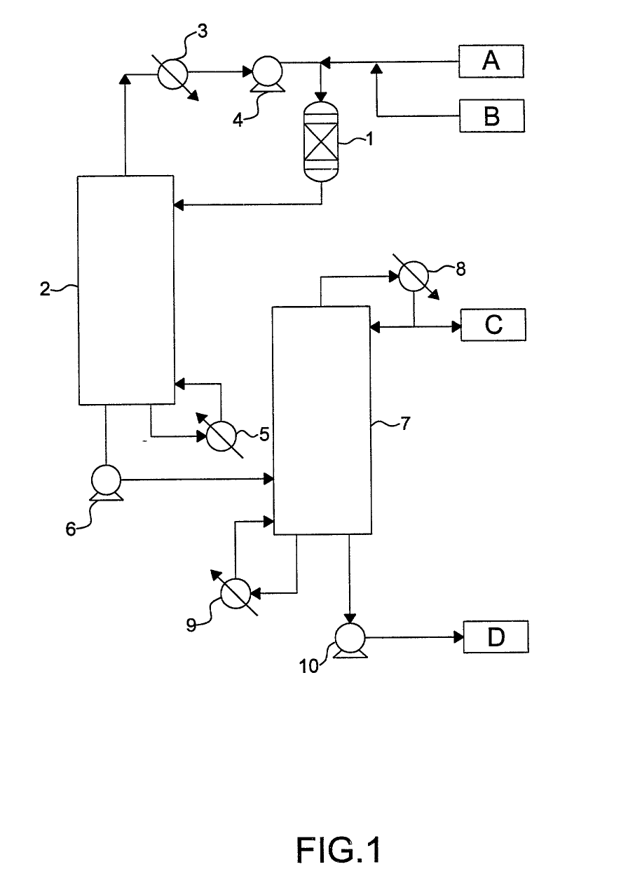

FIG. 1 is a schematic flow diagram of the process of this invention. The main body of the process consists of a reactor (1), a MA separation tower (2) and an acetic acid recovery tower (7). A condensing line, which consists of a condenser (3), a pump (4) and piping, connects the reactor (1) and the MA separation tower (2). Another pump (6) connects the MA separation tower (2) and the acetic acid recovery tower (7). A reboiler (5) is attached at the bottom of the MA separation tower (2). The acetic acid recovery tower (7) is connected to a methanol reservoir (C) and an acetic acid reservoir (D). A methanol condenser (8) is installed between the acetic acid recovery tower (7) and methanol reservoir (C). An acetic acid effluent pump (10) is installed between the acetic acid recovery tower (7) and the acetic acid reservoir (D). Another reboiler (9) is installed at the bottom of the acetic acid recovery tower (7).

Water (A) and methyl acetate (B) are fed to the condensate line at the top of the reactor (1). The mixture of the reaction products from the reactor (1) is introduced to the top of the MA separation tower (2) to separate and recycle un-reacted methyl acetate. The un-reacted methyl acetate co-evaporates with water as an azeotropic mixture in the tower (2), liquified at the condenser (3) and returned to the reactor (1) by the pump (4). The reaction products, acetic acid and methanol, which fall down to the bottom of the MA separation tower (2) with some water, are transferred to the acetic acid recovery tower (7) by the transfer pump (6) to separate acetic acid and methanol. The temperature of the MA separation tower (2) and the acetic acid recovery tower (7) are controlled with the reboilers of (5) and (9), respectively.

In order to reduce the capital investment costs for this invented process, a reflux drum of a conventional distillation tower is utilized as the reactor (1) that is equipped with a fixed bed of solid catalysts. A support screen is installed at the bottom of the catalyst bed to hold the small beads of the catalyst, the diameters of which are in the range of 0.3 to 1.5 mm. Cation exchange catalysts of Amberlyst® 15, Amberlyst® 35 and Amberlyst® 39 from Rohm and Hass™ and Diaion® PK 208H from Mitsubishi Kasei™ are used as the catalysts.

The desirable optimum temperature for the hydrolysis reaction ranges from 50 to 80° C. and it is controlled by adjusting the temperature of the feed water. The pressure at the top of the reactor is controlled between 1.3 to 7 Kg/cm² (absolute pressure). Residence time of the reactants in the reactor (1) is maintained between 5 to 90 minutes. The mole ratio of water to methyl acetate in the feed streams is maintained within the range of 3.0 to 12.0 by controlling the flow rate of the feed water. The operation conditions described above are preferred to reduce the capital and operating costs such as energy and cooling water costs. Either pure methyl acetate or a mixture of methyl acetate and water is used as the feed of methyl acetate (B).

The hydrolysis reaction occurs in the liquid phase. The products, acetic acid and methanol, along with unreacted methyl acetate and water are sent to the top of the MA separation tower (2). The un-reacted methyl acetate vaporizes with water and is recycled back to the reactor (1) to increase the overall conversion of methyl acetate.

The conventional distillation tower, such as tray columns and packed columns, with theoretical plate numbers of 5 to 15 is used as the MA separation tower (2).

A mixture of acetic acid, methanol and water is transferred to the acetic acid recovery tower (7) for separation of methanol and aqueous acetic acid. The methanol is recovered as the top product and the aqueous acetic acid is recovered as the bottom product of the tower (7). The methanol vapor from the top of the tower (7) is condensed by the methanol condenser (8). A portion of the methanol is refluxed to the tower (7) and the rest is sent to the methanol reservoir (C). The aqueous acetic acid, the bottom product, is pumped to the acetic acid reservoir (D).

The following illustrative examples will demonstrate the main process of this invention under preferred conditions.

### EXAMPLES

### Example 1

A mixture of water and methyl acetate with a water-to-methyl acetate mole ratio of 8.0 was fed to the process. Reaction temperature was maintained at 56° C. and the reboiler temperature of the MA separation tower (2) was 91° C. Diaion® PK208H was used as the catalyst. The results are shown in Table 1.

### Example 2

A mixture of water and methyl acetate with a water-to-methyl acetate mole ratio of 5.0 was fed to the process. Reaction temperature was maintained at 60° C. and the reboiler temperature of the MA separation tower (2) was 89° C. Diaion® PK208H was used for the catalyst. The results are shown in Table 1.

### Example 3

A mixture of water and methyl acetate with a water-to-methyl acetate mole ratio of 6.0 was fed to the process. Reaction temperature was maintained at 60° C. and the reboiler temperature of the MA separation tower (2) was 89.9° C. Amberlyst® 15 was used for the catalyst. The results are shown in Table 1.

TABLE 1

| Items | Example 1 | Example 2 | Example 3 |
|---|---|---|---|
| Flow Rate of Methyl Acetate (kg/hr) | 0.2668 | 0.2668 | 0.2668 |
| Flow Rate of Water (kg/hr) | 0.5184 | 0.324 | 0.3888 |
| Mole Ratio (Water/Methyl Acetate) | 8.0 | 5.0 | 6.0 |
| Reboiler Temperature (° C.) | 91.0 | 89.0 | 89.9 |
| Number of Theoretical Plates of the | 10 | 10 | 10 |
| MA Separation Tower | | | |
| Overall Conversion (mole %) | 99.6 | 99.7 | 99.8 |
| Concentration of Acetic Acid (wt %) | 27.4 | 36.48 | 32.9 |
| Concentration of Methanol (wt %) | 14.6 | 19.45 | 17.6 |
| Concentration of Methyl Acetate | 0.125 | 0.135 | 0.08 |
| (wt %) | | | |

Obviously, additional modifications and variations of this invention are possible based on the above teachings. Therefore, it is to be understood that within the scope of the appended claims, this invention may be practiced otherwise than specifically described herein.

What is claimed is:

1. A novel process for the methyl acetate hydrolysis which comprises; 1) a reaction process producing reaction products, acetic acid and methanol, in a reactor packed with a cation exchange resin catalyst from methyl acetate and water which are simultaneously introduced into the condensate line connected to the reactor; 2) a separation process of the above-stated reaction products by using a distillation tower, the MA separation tower, which vaporizes the un-reacted methyl acetate, that is subsequently recycled to the reactor after condensing, and separates both methanol and aqueous acetic acid to the bottom of the MA separation tower ; and 3) a separation process by using the acetic acid recovery tower which from the bottom effluent of the above-stated MA separation tower separates aqueous acetic acid and methanol.

2. The process of claim 1 wherein the mole ratio of methyl acetate to water is in the range of 1.0:3.0 to 1.0:12.0.

3. The process of claim 1 wherein the reactor temperature is in the range of 50 to 80° C.

4. The process of claim 1 wherein the cation exchanged resin catalyst is either one of Amberlyst® 15, Amberlyst® 35, Amberlyst® 39 and Diaion® PK 208H.

5. The process of claim 1 wherein pressure and retention time of the reactor are in the range of 1.3 to 7 kg/cm² and in the range of 5 to 90 minutes, respectively.

6. The apparatus for the methyl acetate hydrolysis process of claim 1 which consists of a reactor, a MA separation tower, an acetic acid recovery tower, condensers, reboilers, reflux drums, pumps, and interconnecting piping as shown in FIG. 1.

7. The apparatus of claim 6 wherein the MA separation tower has 5 to 15 theoretical plates and the acetic acid recovery tower has 5 to 20, theoretical plates.

8. The apparatus of claim 6 wherein the feed lines of water and methyl acetate are connected to the condensate line leading to the reactor.

9. The apparatus of claim 6 wherein the reactor is a conventional reflux drum which is equipped with a fixed catalyst bed and a support screen to prevent leakage of the catalyst.

10. The apparatus of claim 6 wherein the bottom effluent, a mixture of acetic acid, water and methanol, of the MA separation tower is directly utilized in the process of an existing plant without using the acetic acid recovery tower.
